(19) United States
(12) Patent Application Publication
(10) Pub. No.: US 2014/0057109 A1
(43) Pub. Date: Feb. 27, 2014

(54) POLYMER PARTICLES AND METHODS OF MAKING AND USING SAME

(71) Applicant: LIFE TECHNOLOGIES CORPORATION, Carlsbad, CA (US)

(72) Inventors: Steven M. MENCHEN, Fremont, CA (US); Alexander MASTROIANNI, Alameda, CA (US)

(73) Assignee: LIFE TECHNOLOGIES CORPORATION, Carlsbad, CA (US)

(21) Appl. No.: 14/045,601

(22) Filed: Oct. 3, 2013

Related U.S. Application Data
(63) Continuation of application No. PCT/US2012/033084, filed on Apr. 11, 2012.
(60) Provisional application No. 61/473,838, filed on Apr. 11, 2011.

(51) Int. Cl. C12N 15/10

(52) U.S. Cl. CPC C12N 15/1093
USPC 428/402; 526/306; 526/229; 526/200

(57) ABSTRACT

A method of making polymer particles includes making an aqueous gel reaction mixture; forming an emulsion comprising dispersed aqueous phase micelles of gel reaction mixture in a continuous phase at a temperature less than about 10° C.; and performing a polymerization reaction in the micelles. Further, the emulsion comprises at least one polymerization initiator in the micelles of gel reaction mixture. The gel reaction mixture can be maintained at a temperature less than about 10° C. when it comprises the polymerization initiator.

### CROSS-REFERENCE TO RELATED APPLICATION(S)

This application is a continuation application of PCT Application No. PCT/US2012/033084, filed Apr. 11, 2012 and entitled “POLYMER PARTICLES AND METHODS OF MAKING AND USING SAME,”, which claims benefit of U.S. Provisional Application No. 61/473,838, filed Apr. 11, 2011 and entitled “POLYMER PARTICLES AND METHODS OF MAKING AND USING SAME,” which is incorporated herein by reference in its entirety.

### TECHNICAL FIELD

The disclosure, in general, relates generally to methods, compositions, systems, apparatuses and kits for making particle compositions having applications in nucleic acid analysis. Particularly, methods for making polymer particles using emulsions are disclosed.

### BACKGROUND

In order to generate sufficient signal for analysis, many applications in genomics and biomedical research utilize the conversion of nucleic acid molecules in a library into separate, or separable, libraries of amplicons of the molecules, e.g. Margulies et al, Nature 437: 376-380 (2005); Mitra et al, Nucleic Acids Research, 27: e34 (1999); Shendure et al, Science, 309: 1728-1732 (2005); Brenner et al, Proc. Natl. Acad. Sci., 97: 1665-1670 (2000); and the like. Several techniques have been used for making such conversions, including hybrid selection (e.g., Brenner et al, cited above); in-gel polymerase chain reaction (PCR) (e.g. Mitra et al, cited above); bridge amplification (e.g. Shapero et al, Genome Research, 11: 1926-1934 (2001)); and emulsion PCR (emPCR) (e.g. Margulies et al, cited above). Most of these techniques employ particulate supports, such as beads, which spatially concentrate the amplicons for enhanced signal-to-noise ratios, as well as other benefits, such as, better reagent access.

These techniques have several drawbacks. In some cases, amplicons are either in a planar format (e.g. Mitra et al, cited above; Adessi et al, Nucleic Acids Research, 28: e87 (2000)), which limits ease of manipulation or reagent access, or the amplicons are on bead surfaces, which lack sufficient fragment density or concentration for adequate signal-to-noise ratios. In other cases, amplifications must be done in emulsions in order to obtain clonal populations of templates. Such emulsion reactions are labor intensive and require a high degree of expertise, which significantly increases costs.

In the following description, various aspects and embodiments of the invention will become evident. In its broadest sense, the invention could be practiced without having one or more features of these aspects and embodiments. Further, these aspects and embodiments are exemplary. Additional objects and advantages of the invention will be set forth in part in the description which follows, and in part will be obvious from the description, or may be learned by practicing the invention. The objects and advantages of the invention will be realized and attained by means of the elements and combinations particularly pointed out in the appended claims.

### SUMMARY

In some embodiments, the disclosure relates to methods and related compositions, systems, apparatuses and kits for making polymer particles. Particular methods include forming an emulsion including initiator in an aqueous gel phase at a temperature below 10° C.

These above-characterized aspects, as well as other aspects, of the present teachings are exemplified in a number of illustrated implementation and applications, some of which are shown in the figures and characterized in the claims section that follows. However, the above summary is not intended to describe each illustrated embodiment or every implementation of the present teachings.

### BRIEF DESCRIPTION OF THE DRAWINGS

The present disclosure may be better understood, and its numerous features and advantages made apparent to those skilled in the art by referencing the accompanying drawings.

FIG. 1A, FIG. 1B, FIG. 2A, and FIG. 2B include graphs illustrating exemplary populations of particles formed in accordance with the present teachings.

The use of the same reference symbols in different drawings indicates similar or identical items.

### DESCRIPTION OF EXEMPLARY EMBODIMENTS

In some embodiments, the disclosure relates to novel methods of making particle compositions having applications in nucleic acid analysis. More specifically, the disclosure relates to methods of making polymer particles. As used herein, the term “polymer particles,” “non-nucleosidic polymer network,” “polymer network,” “porous microparticle,” and variations thereof, may be used interchangeably and are intended to mean a structure comprising covalently connected subunits, such as monomers, crosslinkers, and the like, in which all such subunits are connected to every other subunit by many paths through the polymer phase, and wherein there are enough polymer chains bonded together (either physically or chemically) such that at least one large molecule is coextensive with the polymer phase, i.e. the structure is above its gel point. In embodiments, the polymer particles may have a volume in the range of from about 65 aL to about 15 pL, or from about 1 fL to about 1 pL.

The polymer networks of the disclosure include those set forth in U.S. Patent Application Publication No. 2010/0304982 A2, which is incorporated herein by reference. Preferably, the polymers of the networks are hydrophilic, they are capable of having a pore or network structure (e.g. average pore diameter, tortuosity, and the like) that permits interior access to various enzymes, especially polymerases, and they are physically and chemically stable under conditions where biomolecules, such as enzymes, are functional and they are substantially non-swelling under the same conditions.

In at least one exemplary embodiment, the polymer network may comprise polyacrylamide gels. Polyacrylamide gels may be formed by copolymerization of acrylamide and bis-acrylamide (“BIS,” N,N′-methylene-bisacrylamide). The reaction is a vinyl addition polymerization initiated by a free radical-generating system. Polymerization may be initiated by ammonium persulfate and optionally TEMED (tetramethylethylenediamine): TEMED accelerates the rate of formation of free radicals from persulfate and these in turn catalyze polymerization. The persulfate free radicals convert acrylamide monomers to free radicals which react with unactivated monomers to begin the polymerization chain reaction. The elongating polymer chains are randomly crosslinked by BIS, resulting in a gel with a characteristic porosity that depends on the polymerization conditions and monomer concentrations. Riboflavin (or riboflavin-5′-phosphate) may also be used as a source of free radicals, often in combination with TEMED and ammonium persulfate. In the presence of light and oxygen, riboflavin is converted to its leuco form, which is active in initiating polymerization, which is usually referred to as photochemical polymerization. In a standard nomenclature for forming polyacrylamide gels, T represents the total percentage concentration (w/v, in mg/mL) of monomer (acrylamide plus crosslinker) in the gel. The term C refers to the percentage of the total monomer represented by the crosslinker. For example, an 8%, 19:1 (acrylamide/bisacrylamide) gel can have a T value of 8% and a C value of 5%.

In various exemplary embodiments, the polymer networks may comprise polyacrylamide gels with total monomer percentages in the range of from about 3% to about 20%, such as in the range of from about 5% to about 10%. In various exemplary embodiments, the crosslinker percentage of monomers may be in the range of from about 5% to about 10%. In additional exemplary embodiments, polymer crosslinker percentage may comprise about 10% total acrylamide, of which about 10% may be bisacrylamide.

Accordingly, in at least one aspect of the disclosure, the polyacrylamide particle composition may comprise a population of polyacrylamide particles with an average particle size of less than about 15 μm, for example less than about 10 μm, or less than about 5 μm, such as 1.5 μm. The polyacrylamide particles may have a coefficient of variation of less than about 20%, for example less than about 15%. In one embodiment, the polyacrylamide particles may have a weight:volume percentage of about 25% or less. In another embodiment, the polyacrylamide particles may be spheroidal and have an average diameter of less than about 3 μm with a coefficient of variation of less than about 20%.

The disclosed methods of making polymer particles comprise the steps of: making an aqueous gel reaction mixture; forming an emulsion comprising dispersed aqueous phase droplets of gel reaction mixture in a continuous phase at a temperature less than about 5° C.; and performing a polymerization reaction in the droplets. In further embodiments, the emulsion comprises at least one polymerization initiator in either the micelles of gel reaction mixture. The gel reaction mixture is maintained at a temperature less than about 10° C. when it comprises the polymerization initiator.

As used herein, the term “aqueous gel reaction mixture,” and variations thereof, is intended to mean an aqueous solution comprising one or more monomers that polymerize under appropriate conditions to form a polymer particle or network as described above. In some embodiments, the aqueous gel reaction mixture optionally includes one or more additional components, e.g., one or more crosslinkers. Optional additional components can include polymerization initiators, such as water soluble polymerization initiators, including those set forth in U.S. Patent Application Publication No. 2010/0304982 A2, such as those in Table I. Optional additional components may also include at least one kind of nucleic acid fragment. Nucleic acid fragments of the disclosure can include nucleic acid primers and DNA fragments from a library, non-limiting examples of which are set forth in U.S. Patent Application Publication No. 2010/0304982 A1. When a nucleic acid fragment is present, the polymer particle may also be referred to as a nucleic acid polymer particle, non-limiting examples of which are also set forth in U.S. Patent Application Publication No. 2010/0304982 A1.

In various embodiments, the aqueous gel reaction mixture may be made by dissolving the monomers and optional additional components in water, such as for example, by combining the monomers with a sufficient amount of water in a conical tube and vortexing the mixture until the monomers are dissolved. When additional components are present in the aqueous gel reaction mixture, they may be dissolved simultaneously with the monomers or separately, either before or after the monomers are dissolved. In an embodiment wherein the aqueous gel reaction mixture comprises polymerization initiators, the polymerization initiators may be dissolved in the mixture after the monomers.

When the aqueous gel reaction mixture comprises a polymerization initiator, the mixture may be maintained at a temperature less than about 10° C., for example less than about 7° C., particularly less than about 5° C. In some embodiments, the mixture may be maintained at a temperature between about 5° C. and about 0° C. In an embodiment, the monomers may be dissolved in water and the solution chilled in an ice bath prior to or during the addition of the polymerization initiator.

An emulsion comprising dispersed aqueous phase micelles of gel reaction mixture in a continuous phase is formed at a temperature less than about 10° C. The emulsion may be formed by dispensing the aqueous gel reaction mixture into a continuous phase while stirring to form droplets.

The continuous phase of the emulsion may comprise at least one oil and at least one surfactant. Examples of oils for use in the continuous phase include, but are not limited to, mineral oil and diethylhexyl carbonate, such as that marketed under the trade name TEGOSOFT DEC® by EVONIK Goldschmidt GmbH of Essen, Germany. Surfactants for use in the continuous phase can include cetyldimethicone copolyol, such as that marketed under the trade name Abil WE09® by EVONIK Goldschmidt GmbH of Essen, Germany.

The continuous phase may further comprise at least one polymerization initiator, such as an oil soluble polymerization initiator, including those set forth in U.S. Patent Application Publication No. 2010/0304982 A2, such as those in Table II.

In various exemplary embodiments where the continuous phase comprises a polymerization initiator, approximately a 3:1 volume ratio of continuous phase to gel reaction mixture may be used to sustain adequate initiator concentration at the oil/water interface during polymerization.

In some embodiments of the disclosed methods, the emulsion is maintained at a temperature of less than about 10° C., typically less than about 7° C., even more typically less than about 5° C. In some embodiments, the emulsion is maintained at a temperature of between about 5° C. and about 0° C. For example, in one embodiment, the aqueous phase may be chilled in an ice bath prior to addition to the continuous phase, and the continuous phase may be in an ice bath during the addition or emulsification.

The emulsion may be degassed after formation while maintaining a temperature of less than about 5° C. Degassing may be performed by gently sparging the emulsion with moistened argon.

The polymerization reaction in the droplets is performed. In an embodiment, the polymerization reaction is initiated by increasing the temperature of the emulsion to a temperature adequate to initiate polymerization, such as about 50° C. or greater, such as 75° C. or greater or to about 90° C. The rate of polymerization initiation depends, in part, upon the temperature of the emulsion.

The methods of the disclosure may further comprise quenching the reaction. For example, quenching can include cooling the emulsion in ice.

The methods of the disclosure may further comprise separating the polymer particles from the continuous phase. For example, separating can include centrifugation, filtering, or other techniques.

In an embodiment, the disclosed methods may produce porous microparticles having three-dimensional scaffolds for attaching greater numbers of template molecules than possible with solid beads that have only a two-dimensional surface available for attachment. In one embodiment, such porous microparticles are referred to herein as nucleic acid polymer particles.

In embodiments, the disclosed methods may produce porous microparticles having shapes with larger surface-to-volume ratios than spherical particles. Such shapes include, for example, tubes, shells, hollow spheres with accessible interiors (e.g. nanocapsules), barrels, multiply connected solids, including doubly connected solids, such as donut-shaped solids and their topological equivalents, triply connected solids and their topological equivalents, four-way connected solids and their topologically equivalents, and the like. Such porous microparticles are referred to herein as “non-spheroidal microparticles.”

In embodiments, the disclosed methods may produce polymer particles at a faster rate than methods known in the art or may yield a greater number of polymer particles from a given batch size than methods known in the art. In at least one embodiment, the method may have a yield of at least 7 trillion particles per batch, compared to 3.6 trillion particles obtained using conventional methods.

In some embodiments, the disclosure also relates to the polymer particles and nucleic acid polymer particles made by the methods disclosed herein.

Additionally, the disclosure relates to the use of the polymer particles disclosed herein in making nucleic acid polymer particles and amplicon libraries, such as described in U.S. Patent Application No. 2010/0304982.

The methods and particles of embodiments of the present teachings provide technical advantages, such as improved time or cost efficient. The methods are capable of producing a high yield of polymer particles, which may also be of high or consistent quality.

### EXAMPLES

### Example 1

### Preparation of 7% Acrylamide/10% Methylene Bisacrylamide/0.3% Ammonium Persulfate Polymer Particle

The following 3 materials are prepared for a one-half scale production of 7% Acrylamide/10% Methylene Bisacrylamide/0.3% Ammonium Persulfate polymer particles:

Oligonucleotide: Dry acrydite tB30 oligonucleotide (10 μmol) is spun down in two 1 mL tubes down to pellet flakes. Then, the flakes are dissolved to 1 mL (10 mM) with water, which utilizes multiple additions of water and dissolution. tB30 is a 30 bp oligonucleotide terminated with PEG and acrydite, available from Eurofins MWG Operon Inc., Huntsville, Ala., USA.

Continuous oil phase (“SNOIL”): 730 mL TEGOSOFT DEC, 200 mL mineral oil, and 70 g Abil WE09 are combined to make 1 L in total volume (90 mL is used for the batch). The oil is not degassed or argon capped. 90 mL of the oil is chilled in a 250 mL heavy weight beaker for at least ten minutes.

Aqueous gel reaction mixture: 0.693 g acrylamide (AA) and 0.077 g methylene bisacrylamide (BIS) are weighed and are placed into a 15 mL conical tube. Approximately 4-5 mL water is added and is vortexed to dissolve. 1.656 mL 10 mM acrydite oligonucleotide (two 0.828 mL portions, one from each tube described above) is added. More water is added up to 11 mL mark. The mixture is chilled in an ice bath for 10 minutes. 0.033 g ammonium persulfate (APS) is weighed, is added to the chilled monomer solution, and is vortexed well, immediately before emulsification.

An emulsion is generated using a Silverson L5M-A solution shearing device fitted with a 1 mm circle grating. The beaker containing the oil phase is placed in an ice bath. The Silverson head is lowered until just in contact with bottom of beaker. The timer is set to 30:30. 10 mL of cold aqueous phase is drawn up in a 10 mL serological pipette. The rotor is started spinning at 2500 RPM, and the rate stabilized. Within the first 30 seconds, the aqueous phase is dispensed directly into the oil near the shaft. While emulsifying, argon is flowed through water.

The emulsion is degassed. The emulsion is transferred into 100 mL glass bottle with a stir bar. The bottle is fitted with a red cap and Teflon-faced septum. The bottle is placed in an ice bath and is stirred on low speed. The cap is pierced with a vent needle and a needle carrying moistened argon from the manifold. The emulsion is sparged gently for 30 minutes with moistened argon, taking care that solution does not blow out of the vent.

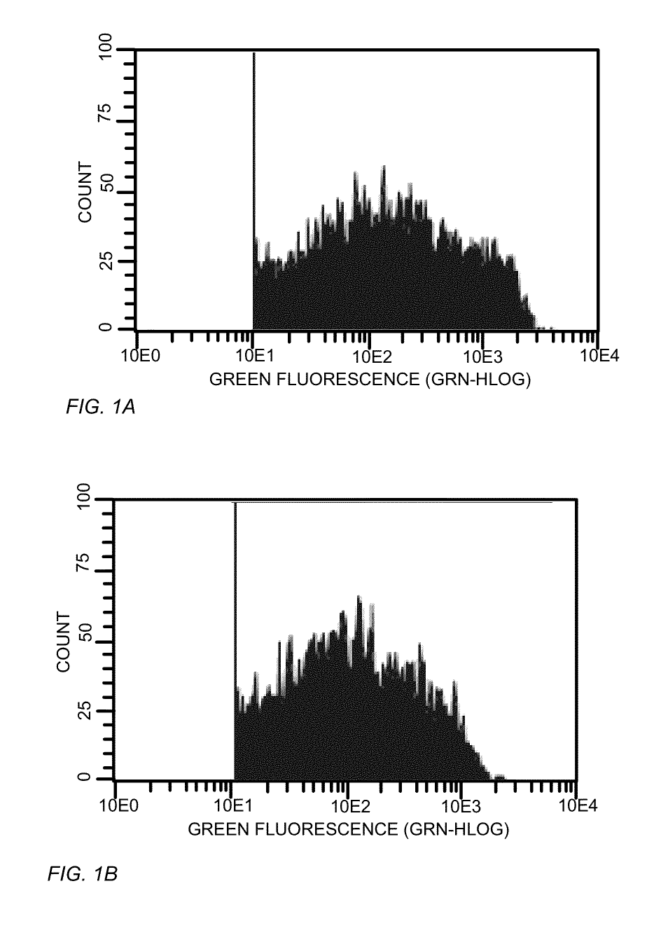

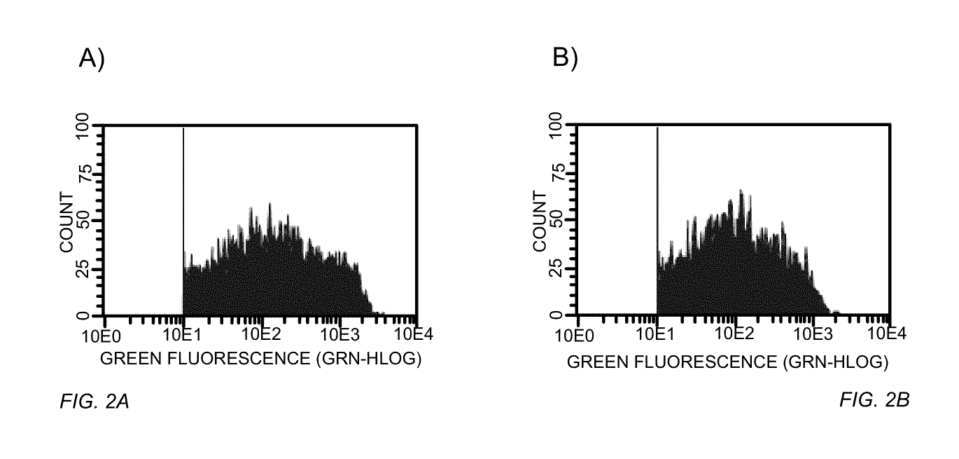

The gel reaction mixture is polymerized. The needles are removed from the cap and the bottle is placed in an oven at 90° C. for 65 minutes, stirring at 750 RPM. The bottle is removed from the oven and is returned to the ice bath where it is stirred gently for 30 minutes to quench the reaction. The total yield is 2.1 trillion particles, as determined by flow cytometry. (See FIG. 1A). As illustrated in FIG. 1, particles prepared by the protocols described in Example 1 are labeled by SYBR Gold staining, are diluted, and are counted using a flow cytometer. Here, the counts, dilution factor, and stock volumes are 714.4 particles/μL, 20,000, and 150 mL. A total particle yield of 2.1 trillion is calculated.

### Example 2

### Preparation of 7% Acrylamide/10% Methylene Bisacrylamide/0.3% Ammonium Persulfate Polymer Particle

The following materials are prepared for a two-times scale production of 7% Acrylamide/10% Methylene Bisacrylamide/0.3% Ammonium Persulfate polymer particles:

Oligonucleotide: Dry tB30 acrydite oligonucleotide (10 μmol) is spun down in seven 1 mL tubes down to pellet flakes. The flakes are dissolved to 1 mL (10 mM) with water, which utilizes multiple additions of water and dissolution. tB30 is a 30 bp oligonucleotide terminated with PEG and acrydite, available from Eurofins MWG Operon Inc., Huntsville, Ala., USA.

Continuous oil phase (“SNOIL”): 730 mL TEGOSOFT DEC, 200 mL mineral oil, and 70 g Abil WE09 are combined to make 1 L in total volume (360 mL is used for the batch). The oil is not degassed or argon capped. 360 mL of the oil is chilled in a 600 mL heavy weight beaker for at least ten minutes.

Aqueous gel reaction mixture (makes 45 mL, 40 mL is used): 2.835 g acrylamide (AA) and 0.315 g methylene bisacrylamide (BIS) are weighed and are placed into a 50 mL conical tube. Approximately 5 mL of water is added and is vortexed to dissolve. 6.77 mL 10 mM acrydite oligonucleotide (from the seven tubes described above) is added. More water is added up to 45 mL mark. The mixture is chilled in an ice bath for 10 minutes. 0.180 g ammonium persulfate (APS) is weighed, is added to the chilled monomer solution, and is vortexed well, immediately before emulsification.

An emulsion is generated using a Silverson L5M-A solution shearing device fitted with a 1 mm circle grating. The beaker containing the oil phase is placed in an ice bath. The Silverson head is lowered until just in contact with bottom of beaker. The timer is set to 30:30. 50 mL of cold aqueous phase is drawn up in a 50 mL serological pipette. The rotor is started spinning at 2500 RPM, and the rate is stabilized. Within the first 30 seconds, the aqueous phase is dispensed directly into the oil near the shaft. While emulsifying, argon is flowed through water.

The emulsion is degassed. The emulsion is transferred into a 500 mL glass bottle with a stir bar. The bottle is fitted with a red cap and Teflon-faced septum. The bottle is placed in an ice bath and is stirred on low speed. The cap is pierced with a vent needle and a needle carrying moistened argon from the manifold. The emulsion is sparged gently for 30 minutes with moistened argon, taking care that solution does not blow out the vent.

The gel reaction mixture is polymerized. The needles are removed from the cap and the bottle is placed in an oven at 90° C. for 65 minutes, stirring at 750 RPM. The bottle is removed from the oven and is returned to the ice bath where it is stirred gently for 30 minutes to quench the reaction. The total yield is 8.7 trillion particles, as determined by flow cytometry. (See FIG. 1B). As illustrated in FIG. 1B, particles prepared by the protocols described in Example 2 are labeled by SYBR Gold staining, are diluted, and are counted using a flow cytometer. Here, the counts, dilution factor, and stock volumes are 361.6 particles/μL, 40,000, and 600 mL. A total particle yield of 8.7 trillion is calculated. FIG. 2A and FIG. 2B provide a comparison of the results obtained through Example 1 and Example 2. As illustrated, the population distribution is similar for both examples.

In some embodiments the disclosure relates to methods and related compositions, systems, apparatuses and kits for making polymer particles, said methods comprising the steps of: making an aqueous gel reaction mixture; forming an emulsion comprising dispersed aqueous phase droplets of gel reaction mixture in a continuous phase. Optionally, the forming is performed at a temperature less than about 10° C. In some embodiments, the disclosed methods further include performing a polymerization reaction in the droplets. The emulsion optionally comprises at least one polymerization initiator in either the droplets of gel reaction mixture or the continuous phase. In some embodiments, the gel reaction mixture is maintained at a temperature less than about 5° C. during emulsification when it comprises the polymerization initiator.

In some embodiments, the disclosure relates generally to methods for making polymer particles, said method comprising: making an aqueous gel reaction mixture; forming an emulsion comprising dispersed aqueous phase droplets of gel reaction mixture in a continuous phase at a temperature of less than about 10° C.; and performing a polymerization reaction in the droplets; wherein the emulsion comprises at least one polymerization initiator in either the droplets of gel reaction mixture or the continuous phase; and wherein the gel reaction mixture is maintained at a temperature of less than about 10° C. when it comprises the at least one polymerization initiator.

In some embodiments, the forming is performed at a temperature of less than about 7° C., typically less than about 5° C.

In some embodiments, the gel reaction mixture is maintained at a temperature of less than about 7° C., typically less than about 5° C., when it comprises the at least one polymerization initiator.

In some embodiments, the disclosure also relates to polymer particles made by the methods set forth herein, including polyacrylamide and N-substituted polyacrylamide polymer particles, and methods of using the same.

In a first aspect, a method of making polymer particles includes making an aqueous gel reaction mixture, forming an emulsion comprising dispersed aqueous phase droplets of the aqueous gel reaction mixture in a continuous phase at a temperature less than about 10° C., and performing a polymerization reaction in the dispersed aqueous phase droplets, wherein the emulsion comprises at least one polymerization initiator in the dispersed aqueous phase droplets of the aqueous gel reaction mixture.

In an example of the first aspect, the temperature is less than about 7° C. For example, the temperature is less than about 5° C. or the temperature is between about 5° C. and 0° C.

In another example of the first aspect and the above examples, making the aqueous gel reaction mixture includes making at a making temperature less than about 10° C. For example, the making temperature is less than about 7° C. In an example, the making temperature is between about 5° C. and 0° C.

In a further example of the first aspect and the above examples, performing the polymerization reaction includes increasing the temperature to at least 50° C.

In an additional example of the first aspect and the above examples, the method further includes quenching the polymerization reaction. For example, quenching includes quenching in a bath having a quenching temperature of less than 10° C.

In an example of the first aspect and the above examples, the polymer particles include crosslinked polyacrylamide or N-substituted polyacrylamide.

In another example of the first aspect and the above examples, the aqueous gel reaction mixture comprises a nucleic acid fragment.

In a further example of the first aspect and the above examples, the continuous phases comprises the at least one additional polymerization initiator.

In an additional example of the first aspect and the above examples, the polymerization initiator includes ammonium persulfate.

In a second aspect, a polymer particle is obtained by a method including making an aqueous gel reaction mixture, forming an emulsion comprising dispersed aqueous phase micelles of the aqueous gel reaction mixture in a continuous phase at a temperature less than about 10° C., and performing a polymerization reaction in the dispersed aqueous phase micelles, wherein the emulsion comprises at least one polymerization initiator in the dispersed aqueous phase micelles of the aqueous gel reaction mixture.

In an example of the second aspect, the particles are polyacrylamide polymer particles.

In another example of the second aspect and the above examples, the particles have a coefficient of variation of less than 20%.

In a further example of the second aspect and the above examples, the particles have an average diameter of less than about 30 μm.

In an additional example of the second aspect and the above examples, the particles have an average diameter in the range of about 0.5 μm to about 30 μm.

In another example of the second aspect and the above examples, the polymer particles have a total monomer percentage in the range of from about 5% to about 10%

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A method of making polymer particles, said method comprising:
making an aqueous gel reaction mixture;
forming an emulsion comprising dispersed aqueous phase droplets of the aqueous gel reaction mixture in a continuous phase at a temperature less than about 10° C.; and
performing a polymerization reaction in the dispersed aqueous phase droplets;
wherein the emulsion comprises at least one polymerization initiator in the dispersed aqueous phase droplets of the aqueous gel reaction mixture.

2. The method of claim 1, wherein the temperature is less than about 7° C.

3. The method of claim 2, wherein the temperature is less than about 5° C.

4. The method of claim 2, wherein the temperature is between about 5° C. and 0° C.

5. The method of claim 1, wherein making the aqueous gel reaction mixture includes making at a making temperature less than about 10° C.

6. The method of claim 5, wherein the making temperature is less than about 7° C.

7. The method of claim 6, wherein the making temperature is between about 5° C. and 0° C.

8. The method of claim 1, wherein performing the polymerization reaction includes increasing the temperature to at least 50° C.

9. The method of claim 1, further comprising quenching the polymerization reaction.

10. The method of claim 9, wherein quenching includes quenching in a bath having a quenching temperature of less than 10° C.

11. The method of claim 1, wherein the polymer particles include crosslinked polyacrylamide or N-substituted polyacrylamide.

12. The method of claim 1, wherein the aqueous gel reaction mixture comprises a nucleic acid fragment.

13. The method of claim 1, wherein the continuous phases comprises the at least one additional polymerization initiator.

14. The method of claim 1, wherein the polymerization initiator includes ammonium persulfate.

15. A polymer particle obtained by a method comprising:
making an aqueous gel reaction mixture;
forming an emulsion comprising dispersed aqueous phase micelles of the aqueous gel reaction mixture in a continuous phase at a temperature less than about 10° C.; and
performing a polymerization reaction in the dispersed aqueous phase micelles;
wherein the emulsion comprises at least one polymerization initiator in the dispersed aqueous phase micelles of the aqueous gel reaction mixture.

16. The polymer particle of claim 15, wherein the particles are polyacrylamide polymer particles.

17. The polymer particles of claim 15, wherein the particles have a coefficient of variation of less than 20%.

18. The polymer particles of claim 15, wherein the particles have an average diameter of less than about 30 μm.

19. The polymer particles of claim 15, wherein the particles have an average diameter in the range of about 0.5 μm to about 30 μm.

20. The polymer particles of claim 15, the polymer particles have a total monomer percentage in the range of from about 5% to about 10%
